(19) United States
(12) Patent Application Publication
(10) Pub. No.: US 2008/0200377 A1
(43) Pub. Date: Aug. 21, 2008

(54) POLYMER PARTICLES

(76) Inventors: Mikael O. Trollsas, San Jose, CA (US); Thierry Glauser, Redwood City, CA (US); Michael Ngo, San Jose, CA (US); Jinping Wan, San Jose, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/676,175

(22) Filed: Feb. 16, 2007

(51) Int. Cl. A61K 47/48; A61K 47/30; A61K 48/00; A61P 35/00; A61P 29/00; A61K 38/17

(52) U.S. Cl. 514/12; 514/772.3; 514/44

(57) ABSTRACT

A composition including a solution suitable for introduction into a blood vessel comprising particles comprising a treatment agent and a polymeric product of at least one activated diamine and at least one activated dicarboxylic acid. A method including introducing a delivery device into a blood vessel; and introducing a solution into the blood vessel, the solution comprising particles comprising a treatment agent and a polymeric product of at least one activated diamine and at least one activated dicarboxylic acid, wherein the activated diamine comprises a product of a diol and an amino acid. A method including combining a treatment agent and a polymeric product of at least one activated diamine and at least one activated dicarboxylic acid; and forming particles of the combination suitable for delivery through a blood vessel.

### BACKGROUND

1. Field

Percutaneous transluminal therapy.

2. Description

The coronary arteries supply the heart with oxygen-rich blood. When the arteries become narrower, less blood and oxygen will flow to the heart. The result of narrowing arteries is commonly referred to as coronary artery disease (CAD). Lack of sufficient oxygen to the heart may cause angina or a heart attack. Most cases of CAD are due to atherosclerosis (hardening of the arteries), in which fatty deposits, called plaques, build up inside the coronary arteries, restricting blood flow.

Drug therapy is effective for the treatment of stable angina and for slowing progression of coronary artery disease. Unstable angina may require surgical intervention in addition to the therapies given for stable angina.

To date, surgery is usually recommended for patients who have unstable angina that does not respond promptly to medical treatment, who have severe recurrent episodes that last more than 20 minutes, or who have other high risk factors for heart attacks. Surgery is also typically performed in people with severe coronary artery diseases (e.g., severe angina, multi-vessel involvement, evidence of ischemia), particularly if abnormalities are evident in the left ventricle of the heart.

A number of invasive techniques are available for treating coronary artery disease. Two standard surgical procedures are coronary artery bypass grafting and percutaneous transluminal coronary angioplasty (PTCA). Studies have generally reported similar effectiveness in the two procedures, although one or the other may be preferable for specific patients. Angioplasty is less invasive than bypass and initially less expensive, although the post-operative need for medications and the high risk for repeat procedures to reopen the artery reduce the long term difference in cost. With the introduction of drug-eluting stents (DESs), the risk of repeated procedures is significantly lowered. However, bypass is still preferred for certain groups of high risk patients. For example, it is generally the recommended procedure for patients with three or more blocked arteries, or where the left main artery is narrowed by 50 percent or more, or when the diseased portion of the artery is very long. Patients with diabetes used to have a significantly better long term survival rate with bypass surgery than with angioplasty, but this difference is much offset by DES.

### SUMMARY

In one embodiment, a composition is disclosed. The composition includes a solution suitable for introduction into a blood vessel including particles (e.g., microparticles and/or nanoparticles) including a treatment agent and a polymeric product of at least one activated diamine and at least one activated dicarboxylic acid. A representative polymeric product is a poly(ester amide) (PEA), where, for example, the activated diamine is itself a product of a diol and an amino acid. In one embodiment, particles prepared from PEA polymers and copolymers are useful as treatment agent depots for regional therapy programs since the polymers/copolymers can be varied to adjust the release rate of treatment agents, including treatment agents of drugs with various polarities. Suitable treatment agents include, but are not limited to, anti-inflammatory agents and anti-proliferative agents.

In another embodiment, a method is disclosed. The method includes introducing a delivery device into a blood vessel and introducing a solution into the blood vessel. The solution, in one embodiment, includes particles, such as microparticles and/or nanopartices including a treatment agent and a polymeric product of at least one activated diamine and at least one activated dicarboxylic acid, such as a PEA. The delivery device may be a device such as a needle suitable for regional delivery of the solution, a percutaneously inserted transluminal catheter having a delivery port located proximate (e.g., proximal) to a treatment site, a trans atrial or trans-ventricular needle to deliver particles to the pericardium, a sub-xiphoid or trans-thoracic approach to deliver particles to the pericardium.

In another embodiment, a method is disclosed that includes combining a treatment agent and a polymeric product of at least one activated diamine and at least one activated dicarboxylic acid and forming particles of the combination suitable for delivery through a blood vessel. Such particles may be microparticles and/or nanoparticles. Suitable polymers include polyester amid such as a PEA and treatment agents include anti-proliferative agents and anti-inflammatory agents.

### BRIEF DESCRIPTION OF THE DRAWINGS

Features, aspects, and advantages of embodiments will become more thoroughly apparent from the following detailed description, appended claims, and accompanying drawings in which:

FIG. 1 shows an embodiment of a catheter assembly disposed in a blood vessel with a distal end proximal to a treatment site within the blood vessel.

FIG. 2 shows another embodiment of a catheter assembly disposed in a blood vessel and shows a delivery device(s) extending beyond the blood vessel.

FIG. 3 shows a stent coated with an active agent/stimuli-responsive polymer disposed at a location in a blood vessel proximate to a treatment site.

FIG. 4 shows a schematic cross-sectional side view of a catheter assembly in a blood vessel including a porous balloon.

### DETAILED DESCRIPTION

Referring to the above discussion of invasive techniques for treating coronary artery disease, to expand the minimally invasive PTCA procedure to the by-pass patient population, drug therapy may be critical based on the DES success experience. Effectively, delivering anti-inflammatory and anti-proliferative drugs to disease sites therefore becomes necessary. One approach to overcome the difficulty to administer typically poorly water-soluble compounds is their incorporation in polymeric carriers (e.g., micro- and/or nanocarriers). Besides their ability to improve the delivery of water-soluble drugs, micro- and/or nanocarriers have been reported to control drug release, to reduce drug associated adverse effects, to protect the drugs from inactivation before reaching their site of action, and to increase intracellular penetration and enhance the pharmacological activity. Normally, the polymers used for making nanoparticles are bio-degradable and bio-compatible.

In one embodiment, a composition is disclosed. The composition includes, for example, a solution suitable for introduction into a blood vessel including a treatment agent and a polymeric product of at least one activated diamine and at least one activated dicarboxylic acid. A representative polymeric product is a poly(ester amide) (PEA), where, for example, the activated diamine is itself a product of a diol and an amino acid. In one embodiment, particles prepared from PEA polymers and copolymers are useful as treatment agent depots for regional therapy program since the polymers/copolymers can be varied to adjust the release rate of treatment agents, including treatment agents of drugs with various polarities.

In one embodiment, the treatment agent is an anti-inflammatory agent. Examples include, but are not limited to corticosteroids such as mometasone furoate, clobetasol and dexamethasone. In another embodiment, the treatment agent is an anti-fibotic agent. An example of an anti-fibrotic agent includes, but is not limited to, Halofuginone. In another embodiment, the treatment agent is estrogen. An example of an estrogen includes, but is not limited to, 17-β-estradiol. In another embodiment the treatment agent is a statin. Examples of statin agent include, but are not limited to, Atorvastatin, Cerivastatin, Fluvastatin, Lovastatin, Rosuvastatin and Simvastatin. In another embodiment, the treatment agent is an anti-proliferative agent. Examples of anti-proliferative agents include, but are not limited to, sirolimus, everolimus and paclitaxel. In another embodiment, the treatment agent may be a combination of anti-inflammatory and anti-proliferative agents such as everolimus/dexamethasone, zotarolimus/dexamethasone, everolimus/mometsone, everolimus/clobetasol, etc. In still another embodiment, a treatment agent is an agent that would benefit a damaged kidney. In still another embodiment, a treatment agent is an agent that would benefit a damaged blood vessel or an infarcted area. In one sense, a treatment agent is an agent that would benefit a blood vessel or an infarcted area (e.g., tissue) by itself creating new cells and/or components thereof or trigger a repair mechanism. A treatment agent may also include an agent that may benefit a blood vessel or an infarcted area (e.g., tissue) only with the assistance of another exogenous or endogenous agent. Suitable treatment agents include, but are not limited to, growth factors (e.g., vascular endothelial growth factor (VEGF), fibroblast growth factor (FGF), platelet-derived growth factor (PDGF), insulin growth factor (IGF), hepa . . . growth factor (HGF), and cocktails of growth factors)), cellular components, proteins, cytokines, and drugs. The treatment agent may also be a gene, a protein or peptide which promotes a benefit, such as the cholesterol efflux from tissues to the liver for excretion. Suitable treatment agents include, but not limited to, genes that encode an ApoA1 protein, ApoA1 protein, ApoA1 Milano protein, ApoA1 mimetic peptides. The treatment agent may further be a glycosaminoglycan such as hyaluronan.

As noted above, the composition includes a polymeric product of at least one activated diamine and at least one activated dicarboxylic acid. In one embodiment, an activated diamine is a product of a diol and an amino acid. The amino acid is reacted with the diol through its carboxyl group. Suitable diols include aliphatic and alicyclic diols. Examples of aliphatic diols include, but are not limited to, C1-C12 alkanes, olefins, and acetylenes (e.g., butanol, hexandiol, butenediol, etc.). Examples of alicyclic hydrocarbons include, but are not limited to, 1,3-cyclohexandiol, 1,4-cyclohexandiol, and dicyclohexandiol. Other suitable diols include oligoalkylene oxides, such as triethyleneglycol.

Suitable amino acids may include any of the various amino acids including glycine, alanine, valine, leucine, isoleucine, phenylalanine, tyrosine, tryptophan, systine, methionine, serine, threonine, lysine, arginine, and histidine. In another embodiment, suitable amino acids are those that form reactive monomers useful for preparing polymers of reasonable molecular weight. Other useful properties of amino acids are their contribution to glass transition temperatures, which will affect the release rate of the treatment agent. Selection of a suitable amino acid may be to target a particular water uptake (e.g., hyrophobicity/hydrophilicity). For example, amino acids containing hydroxyl, carboxyl, or amine side chains tend to be more hydrophilic than amino acids containing only hydrocarbon side chains. The water uptake property of an amino acid will affect treatment agent miscibility and the swelling of the polymer.

In one embodiment, an activated diamine (e.g., product of diol and amino acid) is formed by reacting an amino group with a sulfonic acid, such as 4-methylbenzene sulfonic acid or p-toluene sulfonic acid (PTSA).

As noted above, the composition includes a polymeric product of an activated diamine and an activated dicarboxylic acid. Suitable dicarboxylic acids include saturated cyclic and acyclic dicarboxylic acids such as oxalic acid, malonic acid, succinic acid, glutaric acid, adipic acid, sebacic acid, and cyclic paraffinic dicarboxylic acids; olefinic dicarboxylic acids such as maleic acid and fumaric acid, and cyclic olefin dicarboxylic acids; aromatic dicarboxylic acids such as phthalic acid, isophthalic acid, terephalic acid; and ether dicarboxylic acids, such as glycols and polyethylene glycols. Dicarboxylic acid monomers are activated by reacting the carbonyl group with a phenol such as p-nitrophenol (to form a p-nitrophenolate) or an hydroxyl imide (to form an n-hydroxysuccinimide ester). Examples of paraffinic and olefinic activated dicarboxylic esters include, but are not limited to, the following:

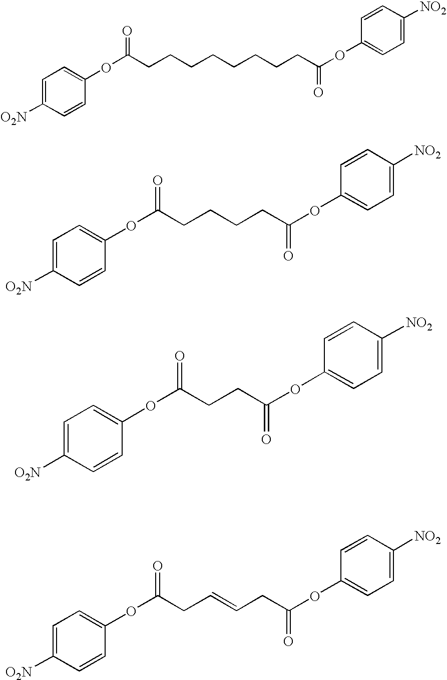

Examples of monomers of a polymeric product of an activated diamine and an activated dicarboxylic acid include, but are not limited to, the following (using leucine, phenylalanine, alanine and valine as the amino acid backbone):

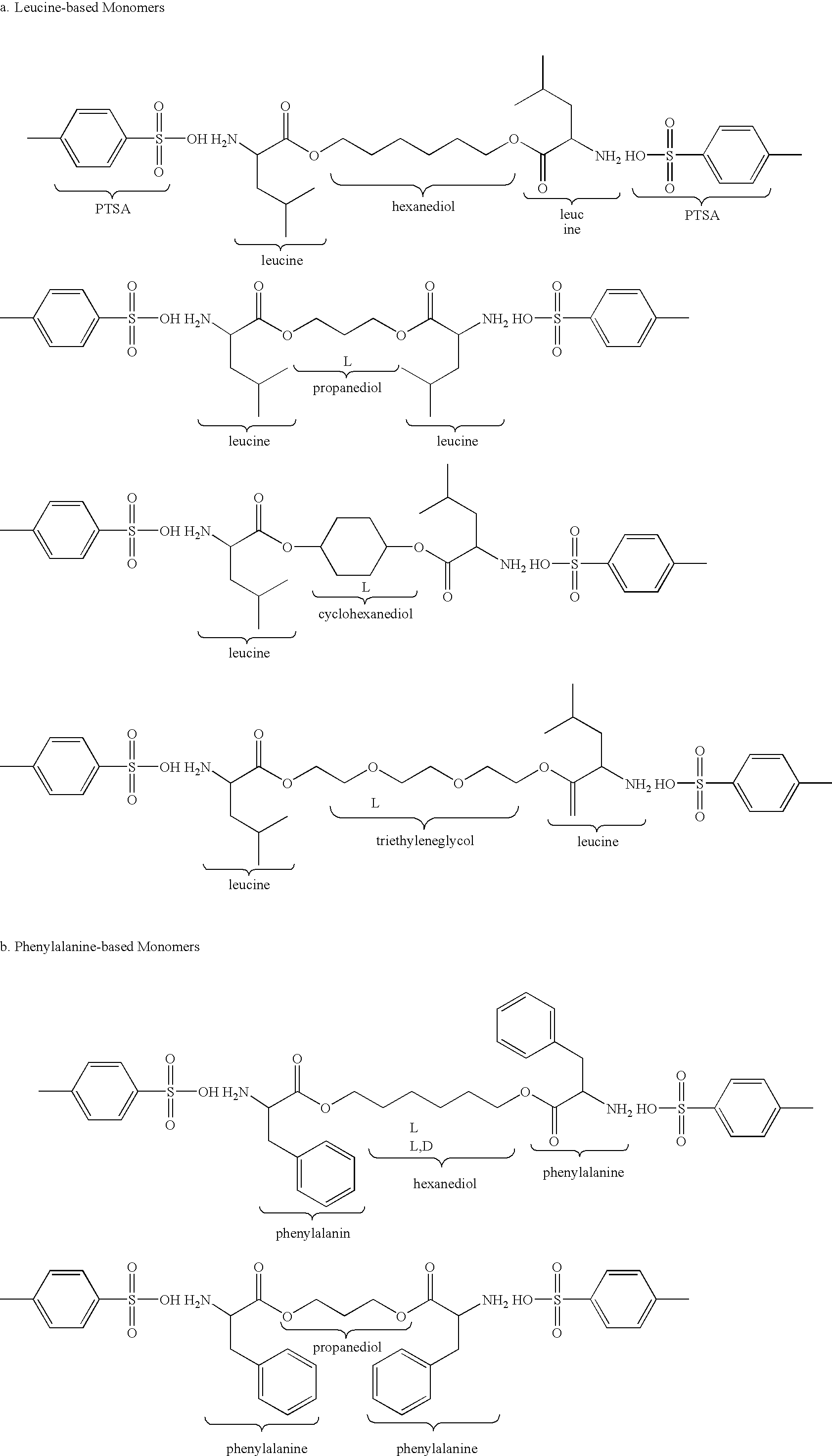

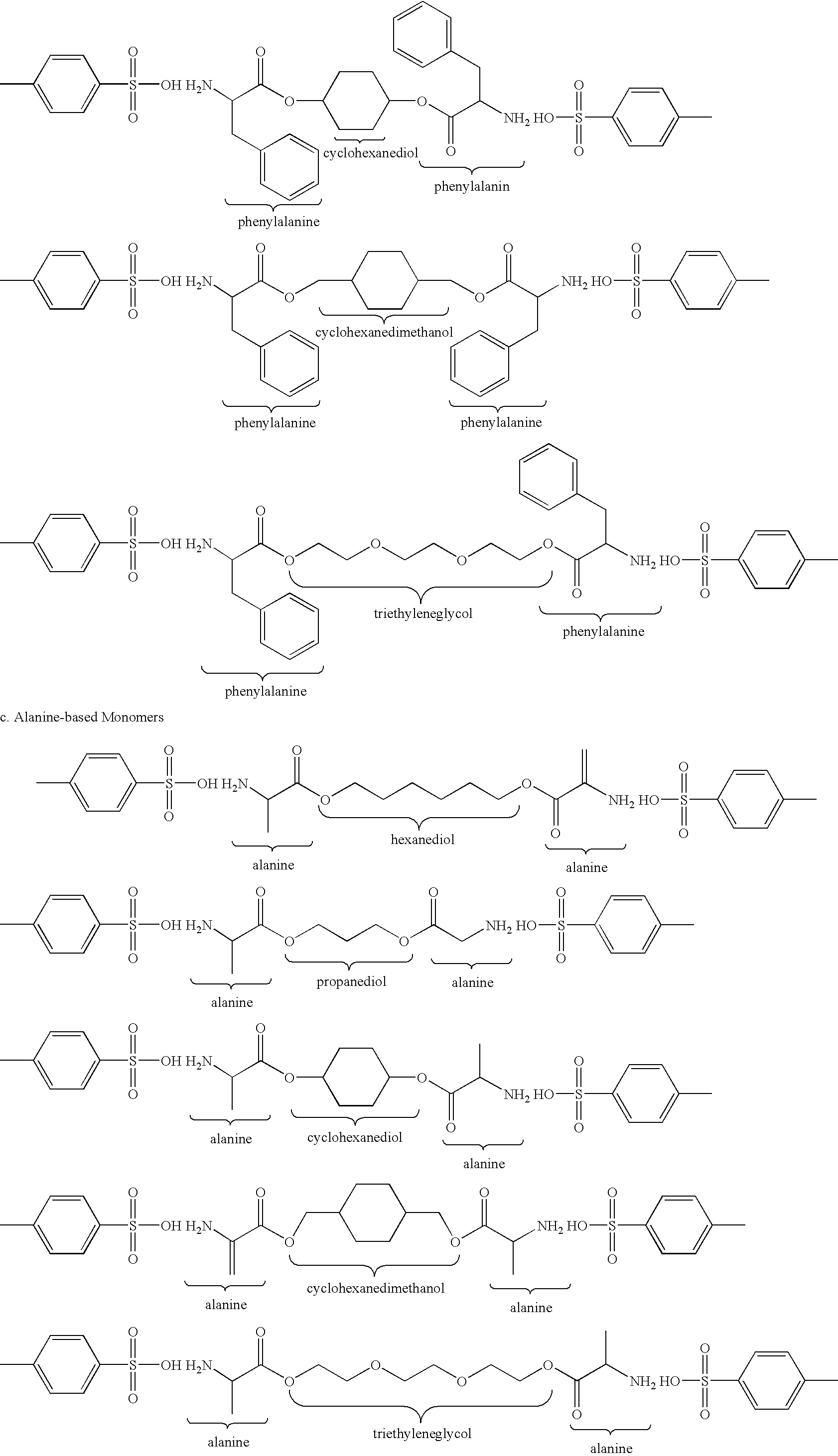

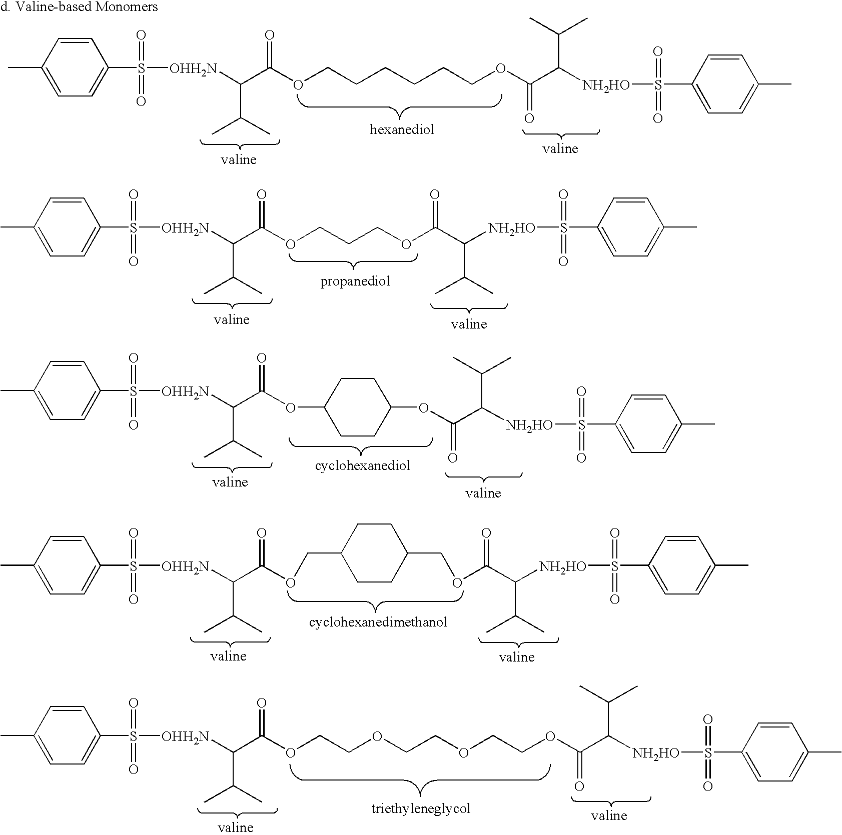

The particles (micro- or nanoparticles) or delivery of a treatment agent may be derived from a polymeric product of one or more activated diamines and one and more activated dicarboxylic acids. In one embodiment, the combination of diamine(s) and dicarboxylic acid(s) is selected to achieve a glass transition temperature (Tg) of the polymeric product that is 37° C. or greater (with 37° C. being representative of the average human body temperature) and, in another embodiment, greater than 37° C. In another embodiment, the polymeric product of one or more activated diamine(s) and one or more activated dicarboxylic acid(s) is selected to achieve a glass transition temperature of 45° C. or greater. Microparticles or nanoparticles derived from polymers having glass transition temperatures on the order of 37° C. or less tend to have a relatively high release rate (permeability) for a treatment agent (i.e., that a treatment agent would tend to be released relatively easily, or sometimes almost immediately) upon introduction into an in-vivo environment such as in a human patient. This may be desirable for certain treatment agents where, for example, it is desirable for immediate release. It may be desirable with other treatment agents, however, to slow the release rate. In such case, the polymer (polymeric product) selected for the micro- or nanoparticles may have a glass transition temperature, Tg, greater than 37° C., such as a Tg of 38° C. to 85° C., with the higher Tg usually indicating a slower release rate for two polymers with similar hydrophilic/hydrophobic balance. Thus, by selection of the polymeric product that is a basis of the particle, the release rate of a treatment agent may be modified. However, a treatment agents release rate is usually primarily controlled by the drug/polymer ratio, the geometry of the polymer matrix, and/or the hydrophilic/hydrophobic balance of the polymer itself.

Examples of polymers and their glass transition temperatures are provided in Table 1:

TABLE 1

| | Tg (° C.) |
|---|---|
| 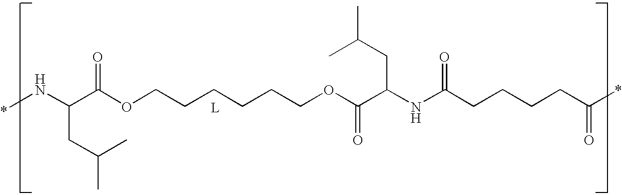 | 36.0 (38) |
| 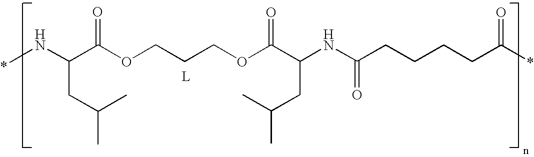 | PEA-11 48.5 (50) |
| 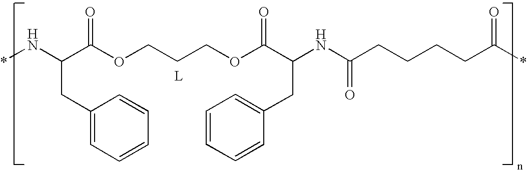 | 53.5 (59) crystalline |
| 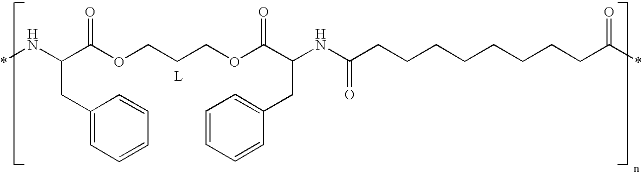 | 52.5 (48) crystalline |
| 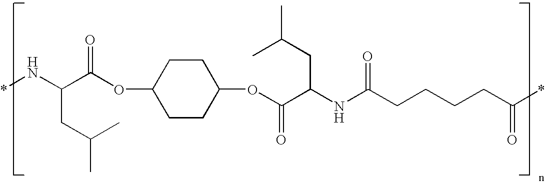 | 82.0 |
| 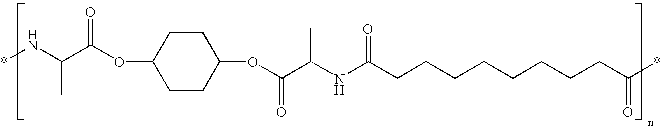 | |
| 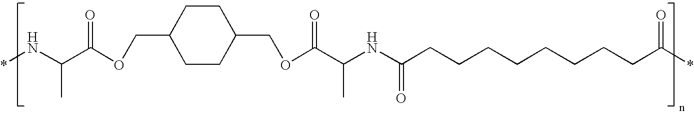 | 47 |
| 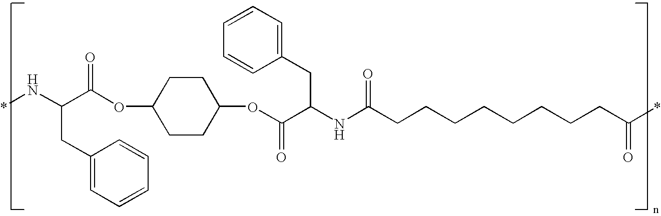 | 74 |
| 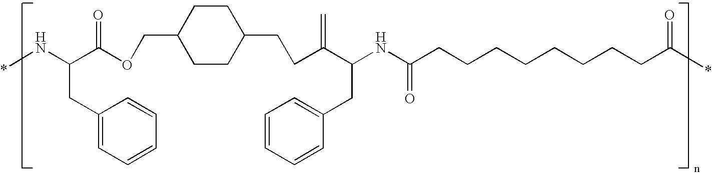 | 55 |
| 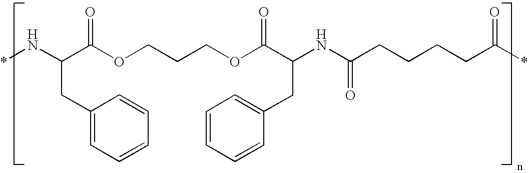 | 53.5 |
| 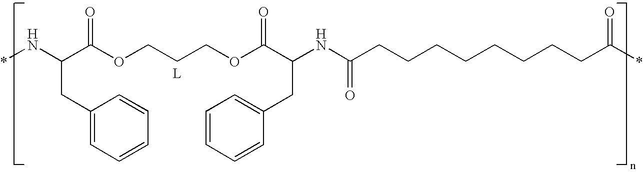 | 42.5 |
| 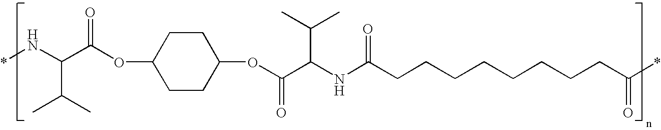 | 75 |

Examples of random copolymers and their glass transition temperatures, are provided in Table 2:

TABLE 2

| | Tg (° C.) |
|---|---|
| 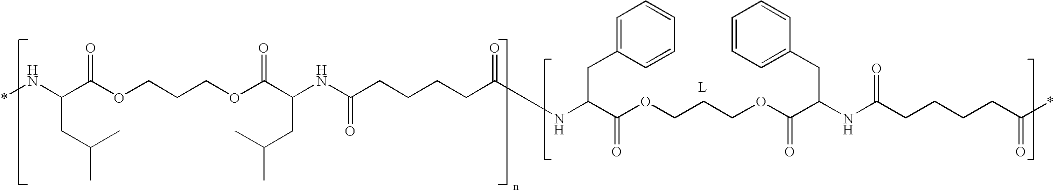 | 45.0crystalline |
| 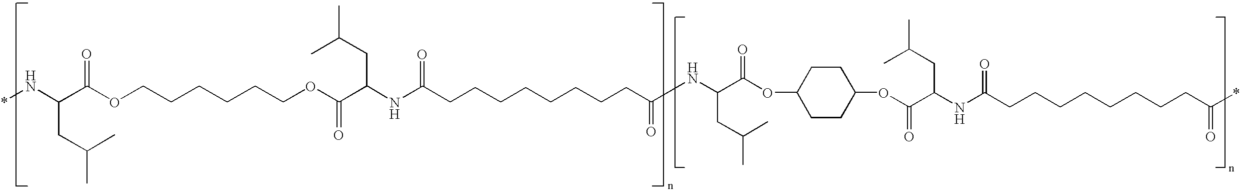 | PEA 4047.3 |
| 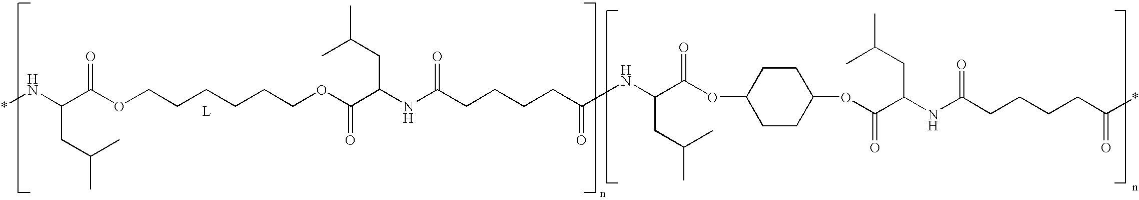 | 45.5 |
| 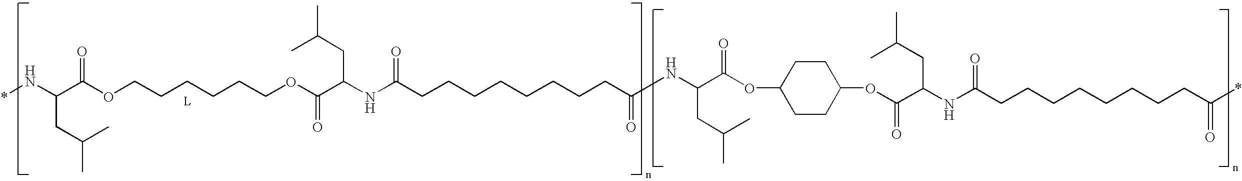 | 31.0 |
| 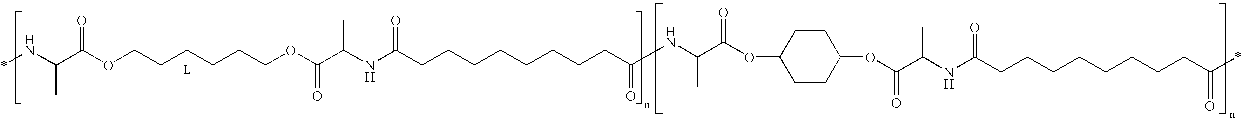 | |
| 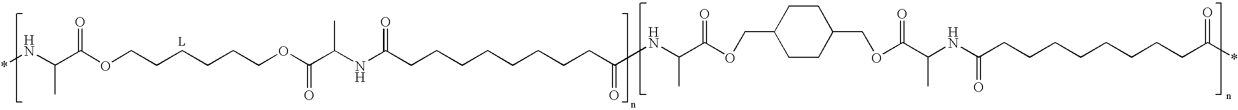 | |
| 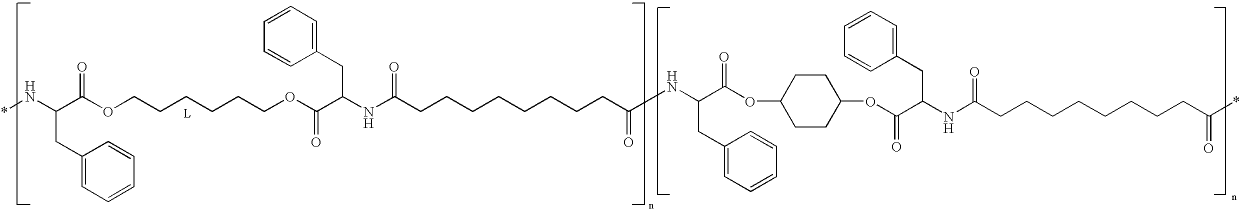 | 50.0 |
| 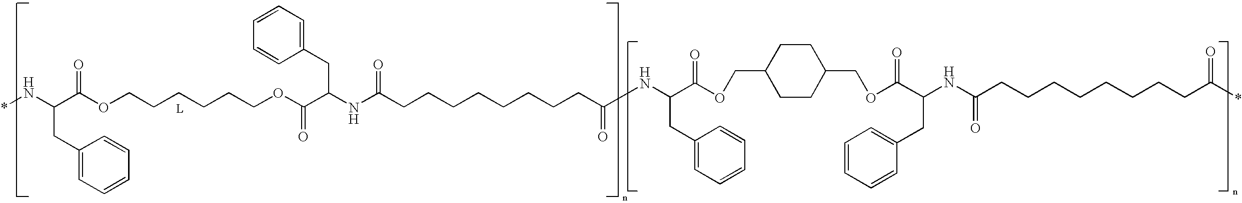 | |

Pure L and D isomers, and racemic mixtures for all amino acids except phenylalanine form amorphous polymers and are therefore equally useful. For phenylalanine the pure isomers L and D can form semi-crystalline polymers and thus the morphology, release rate and degradation rate can be tuned by stereochemistry. A racemic mixture of L and D isomers of phenylalanine and mixtures of L and D isomers form suitable amorphous polymers.

The particles (e.g., micro- and/or nanoparticles) described above may be prepared by spraying a solution of the polymeric moieties and a low volatile solvent or mixture of solvents (e.g., acetone, methylene chloride, trichloroethane, methanol) into a chamber so that the solvent is quickly evaporated and the polymeric product would be left as a small particle. Alternatively the particles can be collected in water, such as solvent evaporation is slow, yielding a smooth dense particle. The size of the particle could be adjusted by polymer concentration, spray rate, and type and setting of a spray nozzle. A treatment agent, such as an anti-inflammatory or anti-proliferative agent (e.g., drug) may also be included in the solution sprayed into the chamber.

Another method for particle preparation is by solvent replacement. One technique would be through the formation of an emulsion. The polymeric moieties are first dissolved in an organic solvent immiscible to water. Examples of such solvents include, but are not limited to dichloromethane, chloroform, and ethylacetate. The polymers are emulsified in an aqueous phase generally containing an emulsifying agent, such as poly(vinyl alcohol), poly(vinylpyrrolidone) and sodium citrate. The solvent is then evaporated off under normal or low pressure to form particles (e.g., micro- and/or nanoparticles). If a hydrophobic treatment agent is to be incorporated with the particles, the hydrophobic treatment agent is dissolved in the organic phase, while water soluble compounds are first dissolved in water and emulsified in the polymer dissolving the organic phase. The primary water-in-oil emulsion thus formed is then processed like the organic polymer phase described above. Alternatively, supercritical fluid (SCF) technology can be used for solvent replacement. SCF technology uses a gas, such as carbon dioxide, in a supercritical state to extract the organic solvent from the forming polymer/treatment agent particle. Advantages to SCF technology include very low residual solvent levels and low processing temperatures.

An alternative solvent replacement method is to dissolve the polymer moieties in organic solvents miscible to water (e.g., acetone, ethanol). The polymers are then dispersed in an aqueous phase generally containing a colloid stabilizer. An example of a suitable colloid stabilizer is poly(vinyl alcohol). An almost instantaneous diffusion of the organic solvent into the aqueous phase results in the precipitation of the polymers as particles. Following the precipitation, the organic solvent may be removed by evaporation or dialysis.

Water soluble treatment agents, such as peptides, proteins, DNA, RNA, small molecules, certain drugs, etc. can be encapsulated in a PEA polymer matrix (e.g., a polymeric product of an activated diamine and an activated dicarboxylic acid) using the methods described above. In one encapsulation study, it was found that PEA microparticles had relatively high drug encapsulation efficiency and relatively constant release of the drug over three weeks.

The ability of PEA microparticles to encapsulate a small peptide has been investigated. It was found that PEA 40 (see Table 2) microparticles with 10% theoretical drug loading resulted in ˜99% encapsulation efficiency. Under the same conditions, the PEA 11 (see Table 1) microparticles had an encapsulation efficiency higher than 85%. Such data indicates that PEA can form a stable emulsion with a treatment agent, resulting in high encapsulation efficiency.

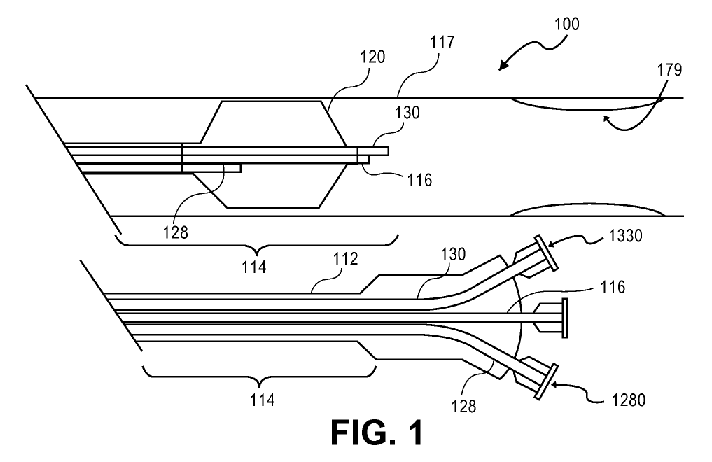

FIG. 1 shows an in vitro release rate study of an ApoA1 peptide from PEA 40 microparticles. FIG. 1 shows that PEA 40 microparticles release the peptide slowly in a sustained manner. Sustained release can last for more than three weeks.

Local delivery of the polymeric micro- and/or nano-particles to the artery tissue to treat restenosis has been investigated. In terms of a suitable particle size for such micro- and/or nano-particles, small size tends (e.g., 50 nm to 200 nm) to be beneficial in terms of device compatibility, sterilization, etc. Larger particles (e.g., 200 nm to 30 μm) will tend to be more desirable if sustained release is concerned. To balance these two concerns, in one embodiment microparticles with 500 nm TO 3 μm size are suitable.

As noted, the PEA family of polymers includes three building blocks: an amino acid, a diol, and a diacid. A suitable PEA polymer may be modified by adding a second different amino acid. By varying the building blocks (e.g., amino acid, diol and diacid), PEA with a wide range of properties may be achieved. The tunability of these polymers is appealing as the polymer properties, such as thermal properties, mechanical properties, melting point, polymer degradation rate, polymer water uptake (e.g., hydrophobicity/hydrophilicity), can be modified to achieve a desired release rate for a treatment agent combined with the polymer. In general, PEAs with a low glass transition temperature (Tg) or that are more hydrophilic will tend to have a higher treatment agent release rate (permeability), while PEAs with high Tg, that are semi-crystalline, that can cross-link, or that are more hydrophobic tend to have lower treatment agent release rate (permeability). Further, the polymer degradation rate can influence the treatment agent release rate if the polymer begins to degrade prior to the complete release of the active agent.

In one embodiment, a polymeric product may include more than one amino acid. For example, one amino acid of an activated diamine may be glycine, valine, alanine, leucine, isoleucine or phenylalanine. An optional second amino acid may be introduced that could include, but is not limited to, lysine, tyrosine, glutamic acid, or cysteine. The second amino acid may contain a reactive side group that would enable the attachment (e.g., conjugation) of pharmacologically active compounds or property modifiers. For example, terminal amine and the sulfhydryl groups of, for example, lysine and cysteine side chains may be exploited to connect drugs, hormones, or other treatment agents. For liposomes and cells, conjugates can generally be prepared by modification through condensation of a protein reactive group (e.g., amine or thiol) on the phosphate group of the liposome. If a treatment agent is conjugated to the PEA backbone, a controlled (e.g., delayed) release can be obtained that will depend on the active agent linker and/or the polymer backbone. Representatively, an activated diamine may include lysine monomers having the following structure:

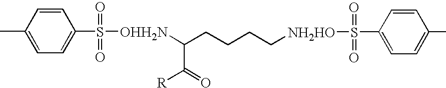

where the R group is an active substance such as a treatment agent (e.g., drug, peptide, etc.) or a polymeric chain/brush. The polymeric/oligomeric chain could be a polyethylene glycol (PEG) or other attachable and water soluble structure or alternatively an attachable hydrophobic polymeric/oligomeric chain or brush. Other potential side groups can be used for imaging purposes, such as a paramagnetic (e.g., MRI) moiety or a contrast agent (e.g., x-ray), or targeting lesions (e.g., peptide sequences that specifically bind to inflammation sites). The linkage for these R groups to the carbonyl of the lysine could be formed through an ester, thioester, amide, anhydride or imide bond, and connected through a spacer of C2-C12 or an alkylene oxide spacer such as a PEG or polypropylene oxide (PPO).

To modify a thermal property of a PEA diols or diacids with cylic structures or double bonds are used. Alternatively the length of the aliphatic carbon chains can be altered, or the use of amino acids with side chains of various structures can be used. Through the use of enantiomerically pure phenylalanine monomers, crystallinity can be introduced into the polymers. Other ways to introduce crystallinity and to change thermal properties is through the use of longer carbon chain linkages, such as aliphatic chains longer than twelve carbon equivalents.

To modify the water uptake (e.g., hydrophobicity/hydrophilicity) of a PEA, a hydrophilic diol or diamine may be conjugated to a PEA to, for example, increase the hydrophilicity which will also tend to increase its active agent release rate (permeability). Examples of hydrophilic diols or diamines include, but are not limited to, polyethylene glycol (PEG), polypropylene glycol (PPG), trietyleneglycol, tetraethyleneglycol, pentaethyleneglycol etc.

Increasing the polymethylene chain length of the diol or diacid building block will tend to reduce the polymer Tg. PEAs based on amino acids with optical rotation (L-isomers) will tend to have a higher Tg than those based on the corresponding D, L-isomers (is this correct?). PEAs synthesized from L-amino acids with smaller or aromatic side substituents (e.g., valine, leucine, phenylalanine) will tend to have higher Tg than those synthesized from L-amino acids with larger aliphaticl side substituents (isoleucine). Amino acids with aromatic moieties (e.g., phenylalanine) will tend to contibute the highest Tg to PEA polymers.

In one embodiment, a composition including microparticles and/or nanoparticles of a treatment agent and a polymeric product of an activated diamine and an activated carboxylic acid (e.g., a particle described by a treatment agent partially or totally encapsulated by the polymer and/or conjugated to the polymer) are formed in a solution suitable for introduction into a blood vessel, such as through an injection needle or catheter. Representatively, an average particle size of suitable microparticles and/or nanoparticles is on the order of ten microns or less.

In general, when PEA polymers are exposed to environments where inflammation is present (such as an active lesion, a vulnerable plaque, a border zone of infarcted tissue or a reperfused area), the polymer degradation rate tends to increase with increased levels of inflammation. Consequently, by leveraging the tunability of the degradation rate, a PEA-based polymer may be selected that exhibits a faster degradation rate when it encounters or is exposed to an inflammatory environment. As an extension, a degradation rate of a PEA polymer can be used to control a treatment agent release rate by, for example, conjugating the treatment agent to the polymer backbone or by ensuring that the polymer begins to degrade prior to the entire treatment agent being released from the treatment agent/polymer matrix. For example, where a treatment agent such as Triamcinolone acetonide (anti-inflammatory corticosteroid) is attached to a lysine of a PEA, the treatment agent may not be released until the polymer degrades.

In one embodiment, the composition may be delivered regionally or locally. A regional delivery may involve, for example, the injection of a composition including microparticles and/or nanoparticles of a treatment agent and a polymeric product of an activated diamine and an activated dicarboxylic acid such as in a saline solution comprising the particles. Thus, a needle injection into a blood vessel (e.g., artery or vein), such as a radial artery or vein is suitable. In this manner, the composition may be introduced to target a treatment site within the blood vessel or beyond the blood vessel. For example, a treatment site may be a stenotic lesion within a blood vessel resulting in occlusion of a blood vessel (e.g., an occlusion of a coronary artery). Alternatively, the treatment site may be a vulnerable plaque within a blood vessel. Alternatively, a systemic delivery may target an ischemic region by, for example, traveling through the circulatory system through capillaries and arterioles associated with an ischemic region. The systemic introduction of the composition requires the composition to travel through the circulatory system to the treatment site. An advantage of PEA as the stimuli-responsive polymer is that PEA tends not to be metabolized as easily as other polymers in the liver and kidney.

In addition to a systemic delivery, the composition may also be delivered locally. FIG. 1 illustrates one embodiment of a delivery apparatus. In general, the delivery apparatus provides a system for delivering a composition, such as stimuli-responsive polymer and an active agent to or through a desired area of a blood vessel (a physiological lumen) or tissue in order to treat a localized area of the blood vessel or to treat a localized area of tissue. The delivery apparatus described is suitable, in one embodiment, for a percutaneous delivery of an active agent where a desired form of the active agent is introduced through a single catheter lumen.

Alternative deliveries would be delivery to the kidney with a specialized catheter, and delivery to the pericardial sac using a trans-ventricular or trans-atrial needle catheter, or accessing the pericardial sac in a sub-xiphoidal or trans-thoracic fashion.

Referring to FIG. 1, the delivery apparatus includes catheter assembly 100, which is intended to broadly include any medical device designed for insertion into a blood vessel or physiological lumen to permit injection and/or withdrawal of fluids, to maintain the patency of the lumen, or for any other purpose. In one embodiment, catheter assembly 100 is defined by elongated catheter body having proximal portion 113 and distal portion 1 14. In one embodiment, proximal portion 113 may reside outside the patient during a procedure while distal portion 114 is placed at a treatment site, for example, within coronary blood vessel 117.

Catheter assembly 100 includes catheter body 112 having a lumen therethrough extending from proximal portion 113 to distal portion 114. In this example, guidewire cannula 116 is formed within catheter body 112 for allowing catheter assembly 100 to be fed and maneuvered over a guidewire (not shown). Guidewire cannula 116 may extend from proximal portion 113 to distal portion 114, thus describing an over the wire (OTW) assembly. In another embodiment, typically described as a rapid exchange (RX) type catheter assembly, guidewire cannula 116 extends only through a portion of catheter body 112, beginning and ending within distal portion 114. It is appreciated that a guidewire may be retracted or removed once catheter assembly 100 is placed at a region of interest, for example, within a blood vessel (e.g., artery or vein). One reason for removing a guidewire may be to utilize guidewire cannula 116 in some additional capacity such as providing a lumen for an imaging assembly or, particularly in an OTW configuration, providing a delivery lumen for a treatment agent.

In the embodiment of FIG. 1, catheter assembly 100 includes balloon 120 incorporated at distal portion 114 of catheter assembly 100 and coupled to a distal end of catheter body 112. Balloon 120 is an expandable body in fluid communication with inflation cannula 128 disposed within catheter body 112. Inflation cannula 128 extends from balloon 120 within distal portion 114 through inflation port 1280 at proximal portion 113 (e.g., at a proximal end of catheter assembly 100). Inflation cannula 128 is used to deliver a fluid to inflate balloon 120.

In the embodiment shown in FIG. 1, balloon 120 is in an expanded or inflated state that totally occludes blood vessel 117. Balloon 120 is selectively inflatable to dilate from a collapsed configuration to a desired or controlled expanded configuration. Balloon 120 can be selectively inflated by supplying a fluid (e.g., liquid) into a lumen of inflation cannula 128 at a predetermined rate of pressure through inflation port 1280. Likewise, balloon 120 is selectively deflatable to return to a collapsed configuration or deflated profile.

Balloon 120 can be made from any suitable material, including but not limited to, polymers and copolymers of polyolefins, polyamides, polyester and the like. The specific material should be compatible with an inflation or expansion fluid and must be able to tolerate the pressures that are developed within balloon 120. One suitable material is an elastomeric nylon such as PEBAX™, a condensation polymerized polyether block polyamide. PEBAX™ is a trademark of ATOCHEM Corporation of Puteaux, France.

Balloon 120 may be inflated by the introduction of a fluid (e.g., liquid) into inflation cannula 128 through inflation port 1280 at a point outside the physiological lumen). Liquids including therapeutic and/or diagnostics agents may be used to inflate balloon 120. In one embodiment, balloon 120 may be made of a material that is permeable to such therapeutic and/or diagnostic agents. In this manner, the therapeutic and/or diagnostic agent may permeate through balloon 120 into the lumen of blood vessel 117. To inflate balloon 120, suitable fluid may be supplied into inflation cannula 128 at a predetermined pressure, for example, between about 1 and 20 atmospheres (atm). A specific pressure depends on various factors, such as the thickness of the balloon wall, the material of which the balloon is made, the type of substance employed, and the flow rate that is desired.

Catheter assembly 100, in the embodiment shown in FIG. 1, also includes delivery cannula 130. Delivery cannula 130 extends from a proximal end of catheter assembly 100 (a proximal end of proximal portion 114) beyond a distal end of balloon 120. At a proximal portion of delivery cannula 130, located at a point outside a physiological lumen as illustrated in FIG. 1, is delivery port 1330. Delivery cannula 130 may be made of a polymer material having a lumen suitably sized to deliver a composition therethrough. In one embodiment, a composition including a treatment agent and a stimuli-responsive polymer may be introduced, in the form of a solution of microparticles and/or nanoparticles into delivery cannula 130 through delivery port 1330. In this manner, a composition may be delivered to a lumen of blood vessel 117. In another embodiment, a needle or hypotube may be placed in delivery cannula 130 and the composition may be introduced into a blood vessel through the needle or hypotube.

In the embodiment in FIG. 1, catheter assembly 100 is placed in blood vessel 117 so that balloon 120 and a distal end of delivery cannula 130 is proximal (upstream) from treatment site 119 that may be a lesion or vulnerable plaque. In one embodiment, balloon 120 may be inflated to occlude blood vessel 117 and therefore inhibit blood flow prior to the introduction of a composition through delivery cannula 130. In this manner, the composition is given an opportunity to interact with treatment site 119 without blood flowing through at a physiological flow rate.

In the embodiment illustrated in FIG. 1, the treatment site is a lesion or vulnerable plaque within blood vessel 119. In the other embodiment, the treatment site may be capillaries or arterioles that may be associated with, for example, infarcted tissue. Thus, it may be desirable to deliver a composition including a treatment agent and a stimuli-polymeric product to capillaries associated with or downstream of blood vessel 117. For example, a treatment agent that is a cellular component may be introduced. By interrupting a coronary blood flow with balloon 120, the dwell time of the treatment agent (cells) in the capillaries may be increased. Typically, the more time that cells are in the capillaries, the more opportunity the cells have to adhere to the capillary walls and transmigrate (travel) into the adjacent tissue where they may multiply and differentiate into tissue types that can repair infarcted tissue. It is believed that ischemic and recently infarcted (dead) tissue produce/release chemicals that signal at least some portion of the mononuclear cells in their vicinity to transmigrate toward/to their location. It is believed that the transmigrated cells differentiate and create new tissue. Similarly, other treatment agents (e.g., anti-inflammatory agents, anti-proliferative) may be introduced to target a treatment site that is ischemic tissue using the assembly of FIG. 1.

In another embodiment, an apparatus (a catheter assembly) is described for accurately locating a treatment agent at a location in a wall of a blood vessel (preferably beyond the media layer) or in a peri-adventitial space adjacent to a blood vessel, or areas radially outward from a peri-adventitial space including at tissue locations such as the tissue of the myocardium. It is appreciated that a catheter assembly is one technique for introducing treatment agents and the following description is not intended to limit the application or placement of the treatment agents described above.

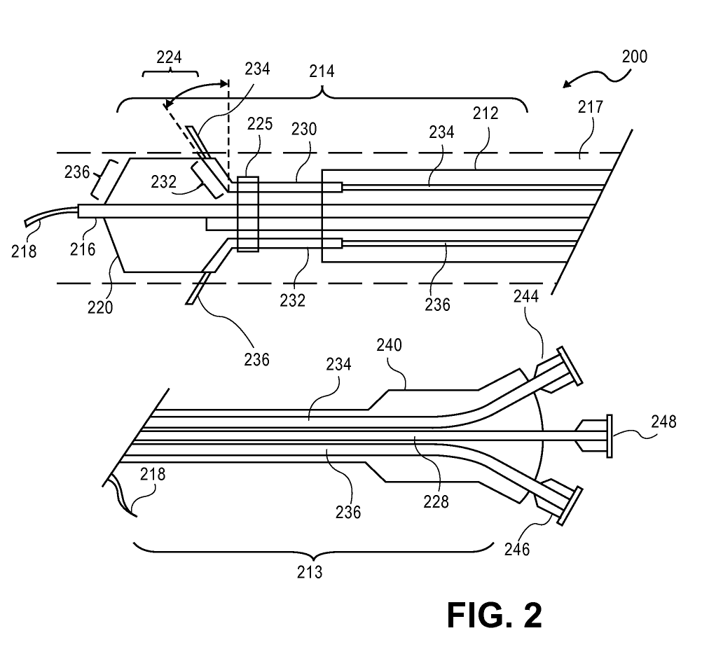

Referring now to the drawings, FIG. 2 illustrates one embodiment of a delivery apparatus. In general, the delivery apparatus provides a system for delivering a composition, such as a solution including micro- and/or nanoparticles of a treatment agent and a stimuli-responsive polymer, to or through a desired area of a blood vessel (a physiological lumen) or tissue in order to treat a localized area of the blood vessel or to treat a localized area of tissue possibly located adjacent to the blood vessel. The delivery apparatus is similar in certain respects to the delivery apparatus described in commonly-owned, U.S. patent application Ser. No. 09/746,498 (filed Dec. 21, 2000), titled “Local Drug Delivery Catheter with Retractable Needle,” of Steward et al.; U.S. patent application Ser. No. 10/394,834 (filed Mar. 20, 2003), titled “Drug Delivery Catheter with Retractable Needle,” of Chow et al.; and U.S. patent application Ser. No. 10/749,354 (filed Dec. 31, 2003), titled “Needle Catheter,” of Chan, et al. The delivery apparatus described is suitable, in one embodiment, for a percutaneous delivery of a treatment agent/particle where a desired form of the treatment agent/particle is introduced through a single catheter needle.

Referring to FIG. 2, the delivery apparatus includes catheter assembly 200, which is intended to broadly include any medical device designed for insertion into a blood vessel or physiological lumen to permit injection and/or withdrawal of fluids, to maintain the patency of the lumen, or for any other purpose. In one embodiment, catheter assembly 200 is defined by elongated catheter body (cannula) 212 having proximal portion 213 and distal portion 214. In one embodiment, proximal portion 213 may reside outside a patient during a procedure while distal portion 214 is placed at a treatment site, for example, within coronary blood vessel 217.

Catheter assembly 200 includes catheter body 212 having a lumen therethrough extending from proximal portion 213 to distal portion 214. In this example, guidewire cannula 216 is formed within catheter body 212 for allowing catheter assembly 200 to be fed and maneuvered over a guidewire (guidewire 218 shown at this point within a lumen of guidewire cannula 216). Guidewire cannula 216 may extend from proximal portion 213 to distal portion 214, thus describing an over the wire (OTW) assembly. In another embodiment, typically described as a rapid exchange (RX) type catheter assembly, guidewire cannula 216 extends only through a portion of catheter body 212, for example, beginning and ending within distal portion 214. An RX type catheter assembly is shown. It is appreciated that guidewire 218 may be retracted or removed once catheter assembly 200 is placed at a region of interest, for example, within a blood vessel (e.g., artery or vein).

In the embodiment of FIG. 2, catheter assembly 200 includes balloon 220 incorporated at distal portion 214 of catheter assembly 200. Balloon 220 is an expandable body in fluid communication with inflation cannula 228 disposed within catheter body 212. Inflation cannula 228 extends from balloon 220 within distal portion 214 through inflation port 248 at proximal portion 213 (e.g., at a proximal end of catheter assembly 200). Inflation cannula 228 is used to deliver a fluid to inflate balloon 220.

In the embodiment shown in FIG. 2, balloon 220 is in an expanded or inflated state that occludes blood vessel 217. Balloon 220 is selectively inflatable to dilate from a collapsed configuration to a desired or controlled expanded configuration. Balloon 220 can be selectively inflated by supplying a fluid (e.g., liquid) into a lumen of inflation cannula 228 at a predetermined rate of pressure through inflation port 248. Likewise, balloon 220 is selectively deflatable to return to a collapsed configuration or deflated profile.

In one embodiment, balloon 220 can be defined by three portions: distal taper wall 226, medial working length 224, and proximal taper wall 222. In one embodiment, proximal taper wall 222 can taper at any suitable angle θ, typically between about 15° to less than about 90°, when balloon 220 is in an expanded (inflated) configuration.

Balloon 220 can be made from any suitable material, including, but not limited to, polymers and copolymers of polyolefins, polyamides, polyester and the like. The specific material employed should be compatible with inflation or expansion fluid and must be able to tolerate the pressures that are developed within balloon 220. One suitable material is an elastomeric nylon such as PEBAX™, a condensation polymerized polyether block polyamide. Other suitable materials for balloon 220 include, but are not limited to, a biocompatible blend of polyurethane and silicone, or a styrenic block copolymer (SBC) or blend of SBCs. Distal taper wall 226, medial working length 224, and proximal taper wall 222 can be bound together by seams or be made out of a single seamless material. A wall of balloon 220 (e.g., at any of distal taper wall 226, medial working length 224 and/or proximal taper wall 222) can have any suitable thickness so long as the thickness does not compromise properties that are critical for achieving optimum performance. Relevant properties include, but are not limited to, high burst strength, low compliance, good flexibility, high resistance to fatigue, the ability to fold, the ability to cross and recross a desired region of interest or an occluded region in a physiological lumen and low susceptibility to defects caused by handling. By way of example, not limitation, a suitable thickness of a balloon wall can be in the range of about 0.0005 inches to 0.002 inches, the specific specifications depending on the procedure for which balloon 220 is to be used and the anatomy and size of the target lumen in which balloon 220 is to be inserted.

Balloon 220 may be inflated by the introduction of a fluid (e.g., liquid) into inflation cannula 228 (through inflation port 248 at a point outside a physiological lumen). Liquids containing therapeutic and/or diagnostic agents may be used to inflate balloon 220. In one embodiment, balloon 220 may be made of a material that is permeable to such therapeutic and/or diagnostic agents. To inflate balloon 220, a suitable fluid may be supplied into inflation cannula 228 at a predetermined pressure, for example, between about 1 and 20 atmospheres (atm). A specific pressure depends on various factors, such as the thickness of the balloon wall, the material of which balloon 220 is made, the type of substance employed, and the flow rate that is desired.

Catheter assembly 200, in the embodiment shown in FIG. 2 also includes delivery cannula 230 and delivery cannula 232 each connected to proximal taper wall 222 of balloon 220 and extending at a proximal end, in one embodiment, into a portion of catheter body 212 of catheter assembly 200. Representatively, a suitable length for delivery cannula 230 and delivery cannula 232 is on the order of three to 6.5 centimeters (cm). Delivery cannula 230 and delivery cannula 232 can be made from any suitable material, such as polymers and copolymers of polyamides, polyolefins, polyurethanes, and the like. Catheter assembly 200, in this view, also includes needle 234 and needle 236. Needle 234 and needle 236 extend from distal portion 214 to proximal portion 213 of catheter assembly 200. At distal portion 214, needle 234 is slidably disposed through a lumen of delivery cannula 230 and needle 236 is slidably disposed through a lumen of delivery cannula 232. Thus, a dimension of delivery cannula 230 and delivery cannula 232 are each selected to be such to allow a delivery device such as a needle to be moved therethrough. Representatively, delivery cannula 230 has an inner diameter (lumen diameter) on the order of 0.002 inches to 0.020 inches (e.g., 0.0155 inches) and an outer diameter on the order of 0.006 inches to 0.05 inches (e.g., 0.0255 inches). FIG. 2 shows catheter assembly 200 with each of needle 234 and needle 236 deployed in an extended configuration, i.e., extending from an end of delivery cannula 230 and delivery cannula 232, respectively. In a retracted configuration, the needles retract proximally into the delivery cannula lumens. Representatively, delivery cannula 230 and delivery cannula 232 may be spaced either radially and/or circumferentially from each other, for example, between 45° and 180° apart.

FIG. 2 shows delivery cannula 230 and delivery cannula 232 each connected to an exterior surface of balloon 220. Specifically, a distal end of each of delivery cannula 230 and delivery cannula 232 extend to a point equivalent to or less than a length of proximal taper wall 222 of balloon 220. One suitable technique for connecting delivery cannula 230 or delivery cannula 232 to balloon 220 is through an adhesive. A suitable adhesive includes a cyanocrylate (e.g., LOCTITE 414™) adhesive, particularly where the balloon material is a PEBAX™ material.

Catheter assembly 200 in the embodiment shown in FIG. 2 also includes sheath ring 225. Sheath ring 225 is positioned over, in this embodiment, guidewire cannula 216, inflation cannula 228, delivery cannula 230, and delivery cannula 232, respectively. In one embodiment, sheath ring 225 functions to inhibit delamination of the delivery cannulas from proximal taper wall 222 of balloon 220 and, where thermally sealed to the various cannulas may reduce the spacing (on a proximal side of sheath ring 225) of the cannulas. Thus, a distal end of sheath ring 225 is placed, in one embodiment, at a point immediately proximal to where a delivery cannula will rotate, bend or plicate in response to the expansion or inflation of balloon 220. In one embodiment, sheath ring 225 is a biocompatible material that is capable of connecting to (e.g., bonding to) a material for balloon 220 and to a material for each of the noted cannulas that it surrounds. Representatively, a body of sheath ring 225 has a length from a proximal end to a distal end on the order of 0.25 millimeters (mm) to 0.75 mm, such as 0.5 mm.

One way to form catheter assembly 200 including sheath ring 225 is to initially connect (e.g., bond) balloon 220 at a distal end to guidewire cannula 216. Balloon 220 is also connected (e.g., bonded) at a proximal end to guidewire cannula 216 and inflation cannula 228. Once balloon 220 is sealed at each end, balloon 220 is inflated. The delivery cannulas are aligned on inflated balloon 220 with a distal end at reference point corresponding to a distal end of proximal taper wall 222 of balloon 220. Distal ends of the delivery cannulas may be tapered to approximate or match a plane defined by medial working length 224 of balloon 220 when balloon 220 is in an inflated state. The delivery cannulas may then be glued or affixed to balloon 220 through an adhesive such as a cyanoacrylate adhesive. Next, sheath ring 225 is loaded (advanced proximal to distal) onto a proximal end of balloon 220 and the cannulas of catheter assembly 200 (e.g., guidewire cannula 216, inflation cannula 228, delivery cannula 230 and delivery cannula 232. A material of sheath ring 225 of a polymer such as PEBAX 40D™ may be connected to balloon 220 and the delivery cannulas by a thermal seal process. As an alternative to a thermal seal process for connecting sheath ring 225, sheath ring 225 may be connected to balloon 220 and the delivery cannulas by an adhesive, such as cyanoacrylate adhesive.

As noted above, each delivery cannula (e.g., delivery cannula 230, delivery cannula 232) plicates or bends distal to sheath ring 225 with the inflation of balloon 220. Thus, the path to be traveled by each needle (e.g., needle 234 and needle 236) includes this bend or plication. To facilitate a travel through a bend or plication region in each delivery cannula and to inhibit puncturing of the respective delivery cannula, each delivery cannula may include a deflector disposed along an interior wall. Representatively, a suitable deflector includes a ribbon of thin, generally flexible and generally resilient material (e.g., thickness on the order of about 0.0005 inches to about 0.003 inches and width on the order of about 0.005 inches and 0.015 inches). Suitable deflectormaterials, dimensions and connections within a catheter assembly are described in commonly-owned, U.S. patent application Ser. No. 09/746,498, filed Dec. 21, 2000 (now U.S. Pat. No. 6,692,466); U.S. patent application Ser. No. 10/394,834, filed Mar. 20, 2003; and U.S. patent application Ser. No. 10/749,354, filed Dec. 31, 2003.

Referring again to FIG. 2, proximal portion 213 of catheter assembly 200 is intended, in one embodiment, to reside outside a patient while the remainder of catheter assembly 200 is percutaneously introduced into, for example, the cardiovascular system of a patient via a brachial, a radial or a femoral artery. In this embodiment, proximal portion 213 of catheter assembly 200 includes hub 240. A similar hub was illustrated with reference to FIG. 1. Hub 240 includes needle 234 and needle 236, and inflation cannula 228. In one embodiment, relative to the materials for the various cannulas described, a housing of hub 240 is a hard or rigid polymer material, e.g., a polycarbonate or acrylnitrile bubadiene styrene (ABS). A distal end of hub 240 has an opening to accommodate a proximal end of catheter body 212. Hub 240 also has a number of cavities at least partially therethrough (extending in a distal to proximal direction) to accommodate needle 234 and needle 236, and inflation cannula 228. A proximal portion of hub 240 flares to separate a spacing between the needles, and inflation cannula 228.

FIG. 2 shows a proximal end of needle 234 and needle 236 each connected (e.g., through an adhesive) to respective injection port 244 and injection port 246. In one embodiment, each injection port includes a luer fitting for conventional syringe attachment. A similar luer fitting may be used in the assembly described with reference to FIG. 1. Each injection port allows for the introduction of a composition, including but not limited to a treatment agent and a polymeric product of an activated diamine and an activated dicarboxylic acid. It is appreciated that a composition introduced at injection portion 244 and injection port 246 may be the same or different (e.g., a composition including a treatment agent and a stimuli-responsive polymer that will be released immediately versus a composition including a treatment agent and a stimuli-responsive polymer in which the treatment agent will be released over time (sustained release); a composition including a treatment agent and a stimuli-responsive polymer in which the treatment agent is an anti-inflammatory agent versus a composition including a treatment agent and a stimuli-responsive polymer in which the treatment agent is an anti-proliferative agent, etc.). In this embodiment, inflation cannula 228 terminates at the distal end of balloon inflation port 248.

In one embodiment, catheter assembly 200 (or catheter assembly 100 in FIG. 1) also includes or can be configured to include an imaging assembly. Suitable imaging assemblies include ultrasonic imaging assemblies, optical imaging assemblies, such as an optical coherence tomography (OCT) assembly, magnetic resonance imaging (MRI).

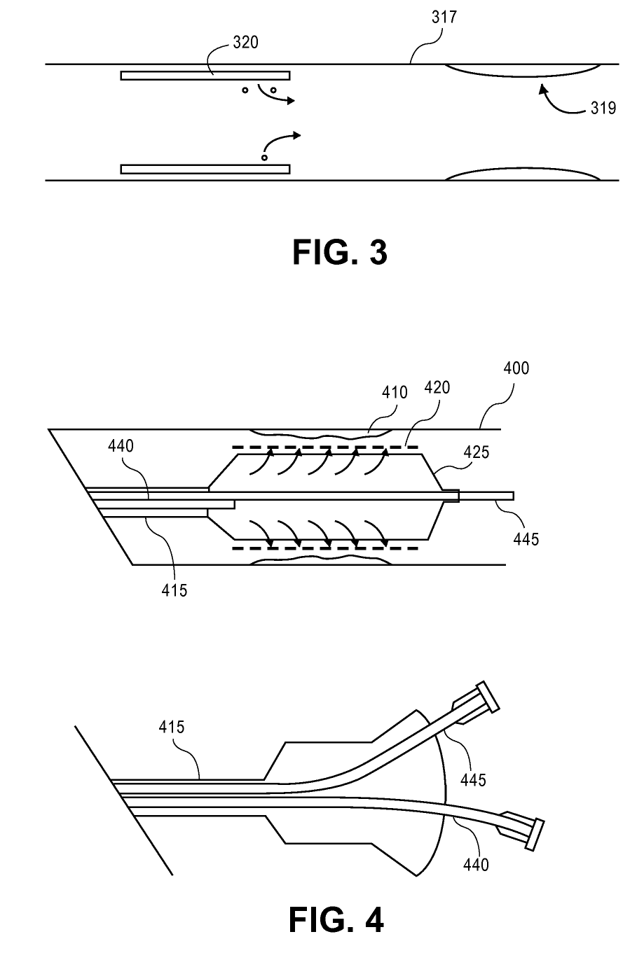

FIG. 3 shows an embodiment of a scaffold (e.g., a stent) placed in a region of a blood vessel including a lesion, such as atherosclerotic plaque. FIG. 3 shows blood vessel 300 including lesion 310 with stent 320 deployed proximal to (e.g., upstream of) the area of plaque 310. Stent 320 may have been deployed in the blood vessel percutaneously by, for example, a stent delivery catheter.

In one embodiment, stent 320 is composed of a metal, an alloy, a polymer, or a combination thereof and a treatment agent included in a stent coating or in the body of the stent. Examples of materials used to form stents include, but are not limited to, ELATINITE®, nitinol (nickel-titanium alloy), stainless steel, tantalum, tantalum-based alloys, platinum, platinum-based alloys, and other metals and their alloys. Alternatively, stent 320 is composed of a bioabsorbable/biodegradable polymer or biostable polymer. A polymer or coating is “bioabsorable” or “biodegradable” when it is capable of being completely or absorbed, substantially degraded or eroded when exposed to either an in vivo environment or an in vitro environment having physical, chemical, or biological characteristics substantially similar to those of the in vivo environment within a mammal. A polymer or coating is “degraded or eroded” when it can be gradually broken down, resorbed, absorbed or eliminated by, for example, hydrolysis, enzymolysis, metabolic processes, bulk or surface erosion, and the like within a mammal.

It is to be appreciated that traces of residue of polymer may remain following biodegradation. A “biostable” polymer is a polymer that is not bioabsorbable.

Suitable polymers used in embodiments of a material for a body stent 320 (i.e., the structural aspect of the stent as opposed to a coating), include, but are not limited to, hydrophobic, hydrophilic, amphiphilic, biodegradable, or a combination thereof. Examples of hydrophobic polymers include, but are not limited to, poly (ester amide), polystyrene-polisobutylene-polystyrene block copolymer (SIS), polystyrene, polyisobutylene, polycaprolactone, poly (L-lactide), poly (D,L-lactide), polylactic acid (PLA), poly (lactide-co-glycolide), poly (glycolide), polyalkylene, polyfluoroalkylene, polyhydroxyalkanoate, poly (3-hydroxybutyrate), poly (4-hydroxybutyrate), poly (3-hydroxyvalerate), poly (3-hydroxybutyrate-co-3-hydroxyvalerate), poly (3-hydroxyhexanoate), poly (4-hydroxyhexanoate), mid-chain polyhydroxyalkanoate, poly (trimethylene carbonate), poly (orthoester), polyphosphohazene, poly (phosphoester), poly (tyrosine derived arylates), poly (tyrosine derived carbonates), polydimethyloxanone (PDMS), polyvinylidene fluoride (PVDF), polyhexafluoropropylene (HFP), polydimethylsiloxane, poly (vinylidene fluoride-co-hexafluoropropylene (PVDF-HFP), poly (vinylidene fluoride-co-chlorotrifluoroethylene) (PVDF-CTFE), poly (butyl methacrylate), poly (methyl mathacrylate), poly (vinyl acetate) (PVA), poly (ethylene-co-vinyl acetate), poly (ethylene-co-vinyl alcohol), poly (ester-urethane), poly (ether-urethane), poly (carbonate-urethane), poly (silicone-urethane), poly (2-hydroxyethyl methacrylate), SOLEF® polyvinylidene fluoride (PVDF), poly (urea-urethane), and combinations thereof.

Examples of hydrophilic polymers include, but are not limited to, polymers and co-polymers of hydroxyethly methacrylate (HEMA); poly (methyl methacrylate) (PMMA); poly (ethylene glycol) acrylate (PEGA); PEG methacrylate; phosphorylcholine; 2-methacryloyloxyethyl phosphorylcholine (MPC); n-vinyl pyrrolidone (VP); carboxylic acid bearing monomers such as methacrylic acid (MA), acrylic acid (AA); hydroxyl bearing monomers such as HEMA, hydroxypropyl methacrylate (HPMA), hydroxypropylmethacrylamide, alkoxymethacrylate, alkoxyacrylate, and 3-trimethylsilylpropyl methacrylate (TMSPMA), poly (ethylene glycol) (PEG), poly (propylene glycol) (PPG), SIS-PEG-polystyrene-PEG, polisobutylene-PEG, PCL-PEG, PLA-PEG, PMMA-PEG, PDMS-PEG, PVD-PEG, PLURONIC® surfactants (poly-propylene oxide-co-polyethylene glycol), poly (tetramethylene glycol), hydroxyfunctinal poly (vinyl pyrrolidone), polyalkylene oxide, dextran, detrin, sodium hyaluronate, hyaluronic acid, heparin, elastin, chitosan; and combinations thereof.

Examples of biodegradable polymers include, but are not limited to, polymers having repeating units such as, for example, an α-hydroxycarboxylic acid, a cyclic diester of an α-hydroxycarboxylic, a dioxanone, a lactone, a cyclic carbonate, a cyclic oxalate, an epoxide, a glycol, an anhydride, a lactic acid, a glycolic acid, a clycolic acid, a lactide, a glycolide, an ethylene oxide, an ethylene glycol, or combinations thereof.

In some embodiments, the biodegradable polymers include, but are not limited, to polyesters, polyhydroxyalkanoates (PHAs), poly (ester amides), amino acids, PEG and/or alcohol groups, polycaprolactones, poly (L-lactide), poly (D,L-lactide, poly (D,L-lactide-co-PEG) block copolymers, poly (D,L-lactide-co-trimethylene carbonate), polyglycolides, poly (lactide-co-glycolide), polydioxanones, polyorthoesters, polyahydrides, poly (glycolic acid-co-trimethylene carbonate), polyphosphoesters, polyphosphoester urethans, poly (amino acids), polycyanoacrylates, poly(trimethylene carbonate), poly (imino carbonate), polycarbonates, polyurethanes, copoly (ether-esters) (e.g., PEO/PLA), polyakylene oxalates, polyphosphazenes, PHA-PEG, and any derivatives, analogs, homologues, salts, copolymers and combinations thereof.

A composition including a treatment agent (or multiple treatment agents) and a stimuli-responsive polymer may be included in a stent coating on stent 320 or included in the body of stent 320 such as, for example, a biodegradable polymeric stent. The release profile of the particles of treatment agent and polymer can be controlled by tailoring the chemical composition and crystallinity of the polymer as the coating or the bioabsorbable stent material (e.g., the more crystalline the slower the release rate).

Table 3 shows a study summary of release rates of various polymers from a VISION® stent (VISION® is a registered trademark of Advanced Cardiovascular Systems of Santa Clara, Calif.). The study compares polymeric products of an activated diamine and an activated dicarboxylic acid, both polymers and copolymers, using either leucine or phenylalanine, as an amino acid backbone with PEA-Tempo:

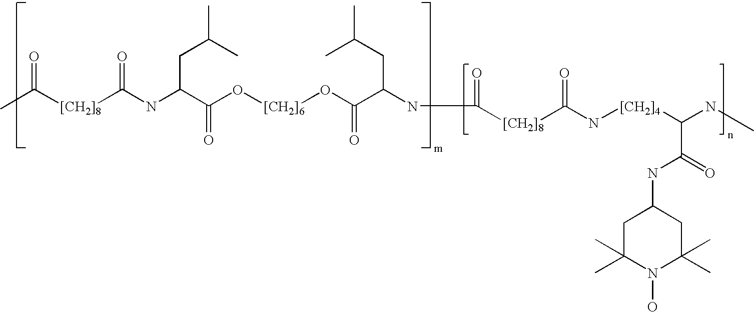

A treatment agent of everolimus was combined with the polymer in a drug to polymer ratio of one to five. Release rates of the drugs from a polymer in porcine serum (PS) were measured at one and three days.

TABLE 3
RELEASE STUDIES (D:P = 1:5)

| | TC | RR-1d | RR-3d |
|---|---|---|---|
| Polymer | (%) | (%) | (%) |
| 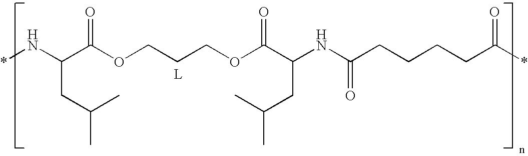 | 94.1 | 95.5 | 99.7 |
| 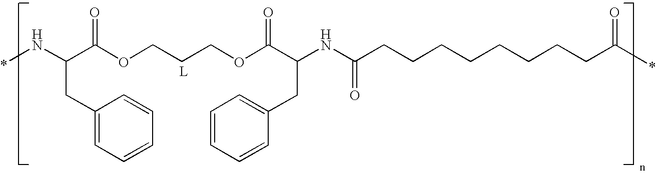 | 91.4 | 44.3 | 64.6 |
| 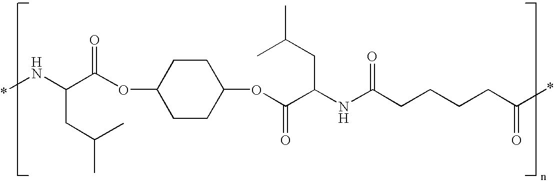 | 94.3 | 0.8 | 7.2 |
| 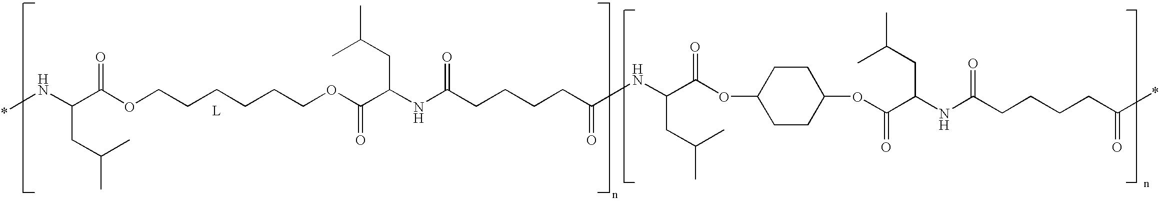 | 92.3 | 41.8 | 70.9 |
| 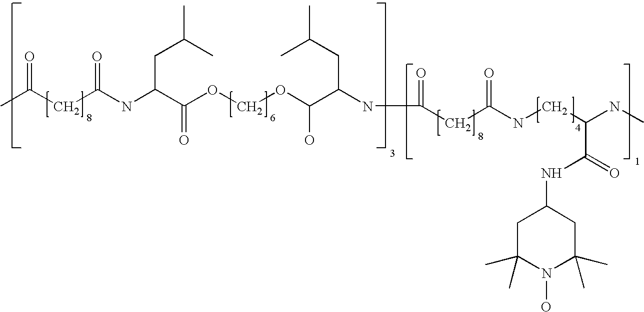 | 92.491.3 | 52.351.7 | 81.582.4 |

Table 4 shows a study summary of release rates of various copolymers from a VISION® stent. The study compares glass transition temperatures and release rates for copolymers including cyclohexane.

TABLE 4
VARIATION OF CYCLOHEXANEDIOL

| Polymer | TC (%) | RR-1d (%) | RR-3d (%) |
|---|---|---|---|
| Hexane/Cyclohexane (Sebacinate) | 75:25 | 31 | n/a |
| | 50:50 | 49 | 20 |
| | 25:75 | 59 | 7 |
| | 0:100 | 83 | 7 |
| Hexane/Cyclohexane (Adipate) | 100:0 | 36 | n/a |
| | 50:50 | 45 | 71 (EtOH) |
| | | | 77 (TCE) |
| | 0:100 | 83 | 7 |
| Tritehyleneglycol/Cyclohexane | 100:0 | 21 (adip.) | n/a |
| | 50:50 | 29 | n/a |
| | 25:75 | 56 | 20 |
| | 0:100 | 83 | 7 |

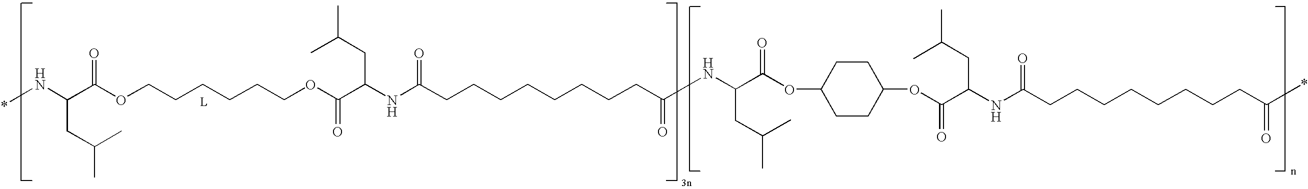

In another embodiment, a composition including treatment agent (or multiple treatment agents) and a polymeric product of an activated diamine and an activated dicarboxylic acid may be delivered locally in combination with stent delivery (e.g., at the time of stent delivery or at a time thereafter). FIG. 4 shows a catheter assembly within a blood vessel. In this embodiment, blood vessel 400 includes lesion 410 such as atherosclerotic plaque or vulnerable plaque. A catheter assembly including balloon 425 is positioned in blood vessel 400 with the balloon at a position adjacent plaque 410. In this embodiment, balloon 425 is shown partially expanded and shown carrying, or acting as a carrier of stent 420. In one embodiment, balloon 425 may be inflated sufficient to place stent 420 against the wall of blood vessel 400, possibly at a position enveloping plaque 410 or at a position proximal to plaque 410. A proximal end or skirt of balloon 425 is connected to a distal end of primary cannula or tube 415 that is, for example, percutaneously, transluminally inserted through, for example, a femoral or radial artery. Primary cannula 415 has a lumen therethrough that includes guidewire cannula 445 (extending in an over-the-wire (OTW) fashion through balloon 425) and inflation cannula 440.

In the embodiment shown in FIG. 4, balloon 425 may be porous so that a treatment agent or a treatment agent or agents in a polymeric product (e.g., nanoparticles or microparticles) may be delivered through pores in the working length of porous balloon 425. Representatively, balloon 425 may be a perfusion balloon of an elastomeric material such as nylon, PEBAX, polyurethanes, or PET with microholes to deliver the bioactive agent. Representatively, microholes may have a diameter on the order of 1 micron to 100 microns. Alternatively, porous balloons constructed from extended polytetrafluoroetylene (ePTFE) such as an EASY STREET® balloon and ultra high molecular weight polyethylene (UHMWPE) with node and fibril structure can be used as a suitable material for balloon 425. Balloon 425 may be thermally bonded to primary cannula 415. Inflation cannula 440 extends from a proximal point exterior to a patient receiving the catheter and a distal end within balloon 425. In this embodiment, a solution containing the treatment agent(s) and polymer particles is delivered through inflation cannula 440 to inflate balloon 425 and deliver the particles into blood vessel 400. In one embodiment, balloon 425 is made of a porous material such as ePTFE. A suitable pore size for an ePTFE balloon material is on the order of one micron to 60 microns. The porosity of ePTFE material can be controlled to accommodate a treatment agent, flow rate or particle size by changing a microstructure of an ePTFE tape used to form a balloon, for example, by wrapping around a mandrel. Alternatively, pore size can be controlled by controlling the compaction process of the balloon or by creating pores (e.g., micropores) using a laser.

In the preceding detailed description, reference is made to specific embodiments thereof. It will, however, be evident that various modifications and changes may be made thereto without departing from the broader spirit and scope of the following claims. The specification and drawings are, accordingly, to be regarded in an illustrative rather than a restrictive sense.

1. A composition comprising:
a solution suitable for introduction into a blood vessel comprising particles comprising a treatment agent and a polymeric product of at least one activated diamine and at least one activated dicarboxylic acid.

2. The composition of claim 1, wherein the activated diamine comprises a product of a diol and an amino acid.

3. The composition of claim 2, wherein the product comprises a single isomer.

4. The composition of claim 1, wherein the polymer further comprises a link of a cyclic aliphatic hydrocarbon.

5. The composition of claim 1, wherein the polymer further comprises a link of a oligoalkylene oxide.

6. The composition of claim 1, wherein the polymer further comprises a link of an unsaturated hydrocarbon.

7. The composition of claim 6, wherein the unsaturated hydrocarbon is a carbonyl.

8. The composition of claim 1, wherein the active agent comprises one of an anti-inflammatory agent and an anti-proliferative agent.

9. The composition of claim 1, wherein the active agent comprises one of genes (DNA, RNA, etc.).

10. The composition of claim 1, wherein the active agent comprises of proteins/peptides such as Apo A1, ApoA1 Milan.

11. The composition of claim 1, wherein the active agent is hyaluronan.

12. The composition of claim 1, wherein the active agent is a growth factor.

13. The composition of claim 1, wherein the active agent is chemically attached to the polymer.

14. The composition of claim 1, wherein the polymer further comprises a lysine monomer.

15. The composition of claim 14, wherein the active agent is chemically attached to the lysine monomer.

16. A method comprising:
introducing a delivery device into a blood vessel; and
introducing a solution into the blood vessel, the solution comprising particles comprising a treatment agent and a polymeric product of at least one activated diamine and at least one activated dicarboxylic acid,
wherein the activated diamine comprises a product of a diol and an amino acid.

17. The method of claim 16, wherein the product comprises a single isomer.

18. The method of claim 16, wherein the polymer further comprises a link of a cyclic aliphatic hydrocarbon.

19. The method of claim 16, wherein the polymer further comprises a link of an oligoalkylene oxide.

20. The method of claim 16, wherein the polymer further comprises a link of an unsaturated hydrocarbon.

21. The method of claim 20, wherein the unsaturated hydrocarbon is a carbonyl.

22. The method of claim 16, wherein the active agent comprises one of an anti-inflammatory agent and an anti-proliferative agent.

23. The method of claim 16, wherein the active agent is chemically attached to the polymer.

24. The method of claim 16, wherein the delivery device comprises a percutaneously inserted transluminal catheter and introducing the delivery device comprises introducing the catheter to a treatment site.

25. A method comprising:
combining a treatment agent and a polymeric product of at least one activated diamine and at least one activated dicarboxylic acid; and
forming particles of the combination suitable for delivery through a blood vessel.

26. The method of claim 25, wherein the particles comprise nanoparticles (replicate for microparticles).

27. The method of claim 25, wherein forming particles comprises spraying a solution comprising the active agent and the polymeric product with a solvent in a drying chamber.

28. The method of claim 27, wherein forming the particles comprises:
dissolving the polymeric product in a solvent;
emulsifying the dissolved polymeric product; and
removing the solvent.

29. The method of claim 27, wherein forming the particles comprises:
dissolving the polymeric product in an organic solvent miscible in water;
dispersing the dissolved polymeric product; and
removing the solvent.

30. The method of claim 27, wherein forming the particles comprises:
dissolving the polymer product and treatment agent in one or more organic solvents to form a solution;
passing the solution through a nozzle to form particles; and
collecting the particles on a substrate or a liquid bath.

31. The method of claim 25, wherein combining comprises conjugating the active agent to the polymer.
